(19) United States
(12) Patent Application Publication
(10) Pub. No.: US 2018/0296531 A1
(43) Pub. Date: Oct. 18, 2018

(54) STIMULATION OF 11B-HSD2 EXPRESSION TO IMPROVE HORMONAL THERAPY OF STEROID-DEPENDENT DISEASE

(71) Applicant: THE CLEVELAND CLINIC FOUNDATION, Cleveland, OH (US)

(72) Inventor: Nima Sharifi, Shaker Heights, OH (US)

(21) Appl. No.: 15/769,112

(22) PCT Filed: Oct. 20, 2016
(86) PCT No.: PCT/US2016/057884

§ 371 Date: Apr. 18, 2018

Related U.S. Application Data
(60) Provisional application No. 62/243,827, filed on Oct. 20, 2015.

(51) Int. Cl. A61K 31/4166; A61P 35/04

(52) U.S. Cl. CPC A61K 31/4166; A61K 45/06; A61K 2300/00; A61P 35/04

(57) ABSTRACT

A method of treating a steroid-dependent disease in a subject is described. The method includes providing steroid hormonal therapy to the subject while inhibiting glucocorticoid receptor activity, or by stimulating the expression of 11 β-HSD2.

### CROSS-REFERENCE TO RELATED APPLICATIONS

This application claims priority to U.S. Provisional Patent Application Ser. No. 62/243,827, filed Oct. 20, 2015, which is incorporated herein by reference.

### GOVERNMENT FUNDING

The present invention was made with Government support under Grant No. 12-038-01-CCE from the American Cancer Society, and Grant Nos. R01CA168899, R01CA172382, and R01CA190289 from the National Cancer Institute. The Government has certain rights in the invention.

### BACKGROUND

Nearly all prostate cancers express the androgen receptor, the importance of which is underscored by androgen deprivation therapy (ADT), the most effective and widely used systemic therapy for prostate cancer for the past 70 years. When combined with radiotherapy, ADT improves survival in selected patients. Similarly, ADT confers a survival advantage if given immediately after prostatectomy in node-positive disease. ADT is the cornerstone of treatment in men with metastatic disease, and has shown benefit even in the setting of biochemical failure after local therapy (i.e., in the context of increasing prostate-specific antigen [PSA] after definitive surgery or radiotherapy). For example, the TOAD trial published in 2016 showed improved survival with early versus delayed ADT in non-metastatic men with increasing PSA level, most of whom had biochemical failure after local therapy. Duchesne et al., Lancet Oncol, 17, 727-37 (2016). Although nearly all men initially show a response to ADT, most will eventually develop castration-resistant prostate cancer. However, the duration of response to ADT varies substantially.

Progression from castration-sensitive to castration-resistant prostate cancer hinges on androgen receptor reactivation, which can occur by several mechanisms. Antonarakis et al. N Engl J Med, 371, 1028-38 (2014). Since the underlying processes typically emerge under selection pressure from ADT, knowledge of such processes generally cannot be used to establish a priori how patients will respond to this treatment. A major advance in the past decade has been the increased appreciation of intratumoural androgen synthesis. Montgomery et al., Cancer Res 68, 4447-54 (2008). Before ADT, tumor androgen supply is dominated by gonadal testosterone. With gonadal suppression during ADT, the serum concentration of testosterone is greatly depleted, inhibiting tumor growth. However, proliferation can continue in the context of intratumoural androgen synthesis, mostly from adrenal precursor steroids and, possibly, at least in part, due to de-novo synthesis from cholesterol. Strong evidence for the importance of intratumoural androgen synthesis is found in the survival benefit from abiraterone (which depletes intratumoural androgens) and enzalutamide (which competes with intratumoural androgens). Scher et al., N Engl J Med, 367, 1187-97 (2012). Additionally, transcripts for several steroidogenic enzymes—including AKR1C3, HSD3B1, and HSD3B2—are consistently upregulated in castration-resistant prostate cancer. Stanbrough et al., Cancer Res, 66, 2815-25 (2006). In 2013, a mutation in HSD3B1 was shown to provide a novel mechanism of resistance to ADT.20 HSD3B1 encodes 3β-hydroxysteroid dehydrogenase-1 (3βHSD1), an isoenzyme that is mainly expressed in peripheral tissues (e.g., the prostate, skin, breast, and placenta), is responsible for catalyzing the rate-limiting step in the conversion of adrenal androgen precursors to dihydrotestosterone, and is required for all pathways of dihydrotestosterone synthesis. Simard et al., Endocr Rev, 26, 525-82 (2005). HSD3B1 (1245A>C) changes aminoacid 367Asn→Thr and renders 3βHSD1 resistant to proteasomal degradation, causing substantial accumulation of this enzyme and, effectively, gain-of-function. The resultant increased intratumoural metabolic flux of adrenal precursors to more potent androgens, such as dihydrotestosterone (the most potent androgen), therefore enhances androgen receptor activation and accelerates tumor proliferation, despite castration.

Emerging data suggest that potent androgen receptor (AR) inhibition with enzalutamide leads to a massive up-regulation of glucocorticoid receptor (GR) expression, which then permits the re-expression of about 50% of AR-responsive genes, in turn promoting tumor progression. Arora et al., Cell 155, 1309-1322 (2013); Isikbay et al., Horm Cancer 5, 72-89 (2014) A challenge has been reconciling these findings with the therapeutic effects of glucocorticoids in castration-resistant prostate cancer. Montgomery et al., Asian journal of andrology 16, 354-358 (2014). Treatment providing a tumor-specific mechanism that regulates GR would therefore be desirable.

### SUMMARY OF THE INVENTION

The present invention provides a method of treating a steroid-dependent disease such as steroid-dependent cancer in a subject by providing steroid hormonal therapy to the subject while inhibiting glucocorticoid receptor activity. In some embodiments, glucocorticoid receptor activity is inhibited by stimulating 11β-HSD2 expression.

The inventors hypothesized that similar to metabolic mechanisms that elicit DHT synthesis, which in turn stimulate AR in castration-resistant prostate cancer (Sharifi, N. Molecular endocrinology 27, 708-714 (2013)), a role for GR in enzalutamide resistance would be accompanied by a tumor metabolic switch that provides sustained tissue cortisol concentrations that enable GR activation. Such a scenario and mechanism may furnish a tumor-specific pharmacologic target and thereby avoid adverse effects associated with systemic GR ablation.

GR stimulation by cortisol in peripheral tissues is physiologically tightly regulated by 11β-hydroxysteroid dehydrogenase-2 (11β-HSD2), which enzymatically converts cortisol to inactive cortisone in humans and corticosterone to 11-dehydrocorticosterone in mice. For example, fetal and placental 11β-HSD2 expression shields against maternal cortisol, thereby restricting GR stimulation and blocking premature fetal maturation. Chapman et al., Physiol Rev 93, 1139-1206 (2013). The inventors show herein that enzalutamide resistance is marked by sustained cortisol concentrations in the prostate tumor that are attributable to a profound loss of 11β-HSD2 and impaired conversion to cortisone, which together de-repress GR and stimulate glucocorticoid-dependent signaling. Mechanistically, 11β-HSD2 loss is mediated by the ubiquitin E3-ligase autocrine mobility factor receptor (AMFR). Finally, sustained 11β-HSD2 expression reverses the metabolic phenotype of enzalutamide resistance and reinstates the therapeutic response to enzalutamide in vivo.

### BRIEF DESCRIPTION OF THE FIGURES

The present invention may be more readily understood by reference to the following figures, wherein:

FIGS. 1A-1D provide schemes and graphs showing that GR stimulation with enzalutamide resistance in prostate cancer is tightly regulated by glucocorticoid metabolism in target tissues. (A) Glucocorticoid metabolism in target tissues. Stimulation of GR by cortisol in humans is limited by 11β-HSD2, which oxidizes and converts cortisol to inactive cortisone. In mice, 11β-HSD2 converts active corticosterone to inactive 11-dehydrocorticosterone. (B) Enzalutamide (Enz) sustains cortisol levels by retarding inactivation in the LAPC4 human prostate cancer cell line. Cells were treated with the indicated concentrations of Enz or vehicle for 36 days, and subsequently treated with [³H]-cortisol (100 nM) for the indicated times, followed by steroid extraction from media (above) and cells (below), steroid separation and quantitation with HPLC. The experiment was done in duplicate and repeated at least 3 times. (C) Cortisol inactivation is impaired in xenograft tumors treated with Enz. Fresh tumor tissues were harvested from LAPC4 xenografts grown in orchiectomized mice and treated with Enz or chow alone (n=5 tumors per treatment group). Tumors were treated with [³H]-cortisol (100 nM) for the indicated times and steroids were extracted from media and analyzed by HPLC. Error bars represent the SD. (D) Enz suppresses LAPC4 cell line proliferation. LAPC4 cells were treated with vehicle (Ctrl) or the indicated concentration of Enz for the designated number of days and cell viability was assessed using CellTiter-Glo. Cell viability was normalized to day 0, experiments were performed in triplicate and error bars represent the SD.

FIGS. 2A-2E provide images showing enzalutamide promotes 11β-HSD2 protein loss in cell line models and tissues from patients with prostate cancer. (A) Enzalutamide (Enz) treatment results in loss of 11β-HSD2 protein that occurs concurrently with an increase in GR protein in the LAPC4 model of CRPC as assessed by Western blot. (B) 11β-HSD2 protein expression in Enz treated LAPC4 cells as assessed by immunocytochemistry. (C, D) Loss of 11β-HSD2 and increase in GR protein similarly occur with Enz treatment in the VCaP and MDA-PCa-2b models. (E) Enz induces loss of 11β-HSD2 protein in tissue from patients with prostate cancer. Local prostate biopsies were obtained from Patients #1 and #2 with image guidance in a neoadjuvant study before (Pre) and after (Post) 2 months of treatment with Enz and medical castration. Patients #3 and #4 had biopsies of metastatic CRPC from lymph nodes before and after 3 months (Patient #3) and 11 months (Patient #4) of treatment with Enz. Fresh tissues from Patients #5-#11 were obtained from surgical prostatectomy specimens and incubated with vehicle or Enz (10 μM) for 7-8 days prior to protein extraction and Western blot.

FIGS. 3A-3C provide graphs showing that 11β-HSD2 expression reverses enzalutamide-sustained cortisol levels and GR-responsive gene expression. (A) Impeded conversion from cortisol to cortisone with Enz treatment is reversible with transient and (B) stable 11β-HSD2 expression. Cells expressing 11β-HSD2 or empty vector (control) were treated with the indicated concentration of Enz for 40 days, followed by treatment with [³H]-cortisol and analysis of steroids in media by HPLC. (C) With Enz treatment, only cortisol-induced GR signaling is specifically reversible with forced stable 11β-HSD2 expression. LAPC4 cells were treated with Enz for 36 days, starved with phenol-red-free medium containing 5% Charcoal:Dextran-stripped FBS for 48 hours and transfected with a plasmid expressing 11β-HSD2 and treated with the indicated conditions for 24 hours. Only cortisol induction of PSA expression, which is GR- and metabolism-dependent, is reversible by 11β-HSD2. Expression of KLF9, which is regulated only by GR, is induced by cortisol and dexamethasone, but only cortisol induction is reversible by 11β-HSD2. Expression of PMEPA1, which is regulated only by AR, is induced with DHT only and not reversible by 11β-HSD2. Expression is normalized to vehicle-treated cells and RPLP0 expression. The experiment was performed 4 times. Error bars represent the SD of a representative experiment performed in triplicate.

FIGS. 4A-4H provide graphs showing that reinstatement of 11β-HSD2 expression restores sensitivity to enzalutamide therapy by specifically suppressing tumor corticosterone. (A) Expression of 11β-HSD2 reverses enzalutamide (Enz) resistant LAPC4 CRPC xenograft tumor growth. (B) Progression-free survival is prolonged by 11β-HSD2 expression in Enz-treated LAPC4 xenografts. N.S.=not significant. (C) 11β-HSD2 expression reverses Enz resistance in the VCaP xenograft model of CRPC as assessed by decreased tumor volume and (D) prolongation of progression-free survival. For both xenograft studies, cells expressing 11β-HSD2 or vector (control) were grown in orchiectomized mice supplemented with DHEA and arbitrarily assigned to Enz or chow (Ctrl). For the comparisons in tumor volume, the significance of the difference between 11β-HSD2/Enz and Vector/Enz was calculated with an unpaired and two-tailed t-test on day 24 (LAPC4) or day 23 (VCaP). For the comparisons in progression-free survival, the significance of the difference between 11β-HSD2/Enz and Vector/Enz was calculated with a log-rank test. (E) The absolute concentration of corticosterone is reduced in xenograft tumors expressing 11β-HSD2. (F) The percentage of corticosterone relative to 11-dehydrocorticosterone is reduced in tumors expressing 11β-HSD2. (G) The absolute concentration of corticosterone and (H) percentage of corticosterone relative to 11-dehydrocorticosterone in serum are unaffected in mice harboring tumors with restored 11β-HSD2 expression. P values in E-H were calculated with an unpaired and two-tailed t-test.

FIGS. 5A-5G provide graphs and images showing AMFR is required for 11β-HSD2 ubiquitination and the enzalutamide-induced metabolic phenotype that sustains local cortisol concentrations and enzalutamide-resistance. (A) 11β-HSD2 and AMFR co-immunoprecipitate. Immunoprecipitation (IP) and immunoblot (IB) from endogenously expressed proteins in whole cell protein lysate from LAPC4 cells were performed with the indicated antibodies. The experiment was performed twice. (B) AMFR promotes 11β-HSD2 ubiquitination. Proteins were expressed in 293 cells, proteins tagged with ubiquitin-His, were pulled-down with Ni-agarose beads, and immunoblot was performed with the indicated antibodies. The experiment was performed twice. (C) Silencing AMFR expression with two independent shRNAs increases 11β-HSD2 protein. LAPC4 cells stably expressed shRNAs against AMFR (shAMFR) or non-silencing control expression vector. The experiment was performed 3 times. (D) Blockade of Enzalutamide (Enz)-mediated 11β-HSD2 loss by silencing AMFR reverses the metabolic phenotype that confers sustained cortisol concentrations. Cells were treated with the indicated concentration of Enz and subsequently were treated with [³H]-cortisol (100 nM) for the indicated times, followed by steroid extraction from media and cells, and steroid analysis by HPLC. Error bars represent the SD of biological triplicates. The experiment was performed 3 times. Enz treatment in panel D was for 38-42 days. (E) AMFR is required for tumor growth through enzalutamide therapy. Xenografts from LAPC4 cells expressing shAMFR or non-silencing control vector (shCtrl) were grown in surgically orchiectomized mice supplemented with DHEA and treated with Enz when tumors reached 100 mm³. The significance of the difference between shCtrl and shAMFR groups was calculated with an unpaired and two-tailed t-test on day 20. (F) Progression-free survival is increased in tumors lacking AMFR. The significance of the difference between shCtrl and shAMFR groups was determined with a log-rank test. (G) Xenograft tumors with genetic ablation of AMFR retain 11β-HSD2 protein expression. Xenograft tissues were collected at the end of the xenograft study and immunoblot was performed with the indicated antibodies.

FIGS. 6A-6C provide graphs showing the effects of Enz on LAPC4 cells. (A) Short term Enz treatment does not affect cortisol metabolism. Previously untreated cells were treated with the indicated concentration of Enz or Vehicle and concomitantly with [³H]-cortisol (100 nM) for the indicated times and steroids were separated and quantitated by HPLC. (B) Enz suppresses expression of AR-regulated transcripts and has no acute effect on expression of GR, HSD11B2 or HSD11B1. LAPC4 cells were treated with the indicated concentration of Enz or Vehicle for 24 hours and the indicated transcripts were assessed by qPCR. Expression is normalized to Vehicle control and RPLP0. (C) Viability of LAPC4 cells recovers with long-term Enz treatment. Cells were treated with long-term (Enz D56), short-term (Ctrl+Enz) Enz (10 μM), or no treatment (Ctrl) for the indicated number of days and cell viability was assessed relative to day 0. Experiments were performed in triplicate and error bars represent the SD.

FIGS. 7A-7F provide graphs and images showing the response to Enz in prostate cancer cell lines and human tissues. (A) Enz treatment does not change 11β-HSD2 protein expression in the AR-negative DU145 prostate cancer cell line. (B) Enz treatment does not change 11β-HSD1 protein expression in AR-expressing prostate cancer cell lines. Cells were treated with the indicated concentrations of Enz, whole cell protein lysates were obtained, separated and assessed with anti-11β-HSD1 and anti-β-actin antibodies. (C) GR transcript increases and HSD11B2 is unchanged with long-term Enz treatment of LAPC4 and VCaP cells. Expression is normalized to vehicle treated cells and RPLP0. (D) Enz does not directly antagonize 11β-HSD2. LAPC4 cells were transfected with a vector encoding 11β-HSD2, in the presence of the indicated concentration of Enz or Vehicle, and conversion from [³H]-cortisol (100 nM) to cortisone was assessed by HPLC. Experiments performed in biological duplicate. (E) 11β-HSD2 loss is not attributable to GR stimulation. LAPC4 cells were treated with dexamethasone (DEX; 100 nM) for the indicated durations, whole cell protein lysates were obtained and assessed with anti-11β-HSD2 and anti-β-actin antibodies. (F) GR protein expression is induced in a subset of the patient tissues from FIG. 2E. All 6 tissues that have induction of GR expression exhibit loss of 11β-HSD2.

FIG. 8 provides an image showing 11β-HSD2 overexpression (OE) in long-term Enz-treated LAPC4 cells is comparable to endogenous expression in the human placental derived JEG-3 cell line.

FIG. 9 provides an image showing forced 11β-HSD2 expression in Enz-treated LAPC4 xenografts is comparable to endogenous expression in the MDA-PCa-2b prostate cancer cell line and the human placental derived JEG-3 cell line.

FIGS. 10A-10E provide graphs and images showing AMFR and Erlin-2 regulation and cortisol metabolism with Enz treatment. (A) Erlin-2 but not AMFR is consistently up-regulated with Enz treatment of LAPC4 cells. (B) Erlin-2 is up-regulated in 8 of 11 human prostate tissues. Immunoblots were performed as described previously. (C) Erlin-2 overexpression (OE) suppresses expression of 11β-HSD2 protein in LAPC4 cells and Erlin-2 knockdown by siRNA increases 11β-HSD2 expression in the long-term Enz-treated LAPC4 cells. (D) AMFR silencing does not regulate HSD11B2 transcript. qPCR was performed in triplicate and expression is normalized to shControl-expressing cells and RPLP0. (E) Reversal of the metabolic phenotype that sustains cortisol with Enz treatment by AMFR knockdown is reversed again by 11β-HSD2 knockdown (compare cortisol at 24 hrs in shAMFR groups between siCTRL and siHSD11B2). The specificity of siHSD11B2 is shown by qPCR and immunoblot. LAPC4 cells stably expressing stably shCTRL or an shAMFR construct were treated with Enz as described, transiently transfected with siHSD11B2 or siCTRL and treated with [³H]-cortisol (100 nM). Experiments were performed in duplicate.

### DETAILED DESCRIPTION OF THE INVENTION

The inventors have demonstrated that glucocorticoid metabolism plays a significant role in the development of resistance to steroid hormonal therapy, and that direct inhibition of glucocorticoid receptor activity, or inhibition of glucocorticoid receptor activity by stimulating 11β-HSD2 expression, can therefore be used to overcome resistance to steroid hormonal therapy.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

Treat”, “treating”, and “treatment”, etc., as used herein, refer to any action providing a benefit to a subject afflicted with a steroid-dependent disease such as prostate cancer, including improvement in the condition through lessening or suppression of at least one symptom, delay in progression of the disease, etc.

Prevention, or prophylaxis, as used herein, refers to any action providing a benefit to a subject at risk of being afflicted with a steroid-dependent disease such as prostate cancer, including avoidance of the development of the condition or disease or a decrease of one or more symptoms of the disease should a disease develop. The subject may be at risk as a result of family history.

“Pharmaceutically acceptable” as used herein means that the compound or composition is suitable for administration to a subject for the methods described herein, without unduly deleterious side effects in light of the severity of the disease and necessity of the treatment.

The term “therapeutically effective” is intended to qualify the amount of each agent which will achieve the goal of decreasing disease severity while avoiding adverse side effects such as those typically associated with alternative therapies. The therapeutically effective amount may be administered in one or more doses. An effective dose, on the other hand, is an amount sufficient to provide a certain effect, such as enzyme inhibition, but may or may not be therapeutically effective.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of heterologous nucleic acid. For example, a transformed cell can include an 11β-HSD2 expression vector to increase 11β-HSD2 expression.

The term “expression vector” as used herein refers to a DNA sequence capable of directing expression of a particular nucleotide sequence in an appropriate host cell. Expression vectors can contain a variety of control sequences (e.g., promoters and terminators), structural genes (e.g., genes of interest), and nucleic acid sequences that serve other functions as well. The construct comprising the nucleotide sequence of interest can be chimeric. The nucleotide sequence of interest, including any additional sequences designed to effect proper expression of the nucleotide sequences, can also be referred to as an “expression cassette”.

The terms “promoter” or “promoter region” each refer to a nucleotide sequence within a gene that is positioned 5′ to a coding sequence and functions to direct transcription of the coding sequence. The promoter region comprises a transcriptional start site, and can additionally include one or more transcriptional regulatory elements. Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell.

The term “operatively linked”, when describing the relationship between two nucleic acid regions, refers to a juxtaposition wherein the regions are in a relationship permitting them to function in their intended manner. For example, a control sequence “operatively linked” to a coding sequence can be ligated in such a way that expression of the coding sequence is achieved under conditions compatible with the control sequences, such as when the appropriate molecules (e.g., promoters and/or terminators) are bound to the control or regulatory sequence(s). Thus, in some embodiments, the phrase “operatively linked” refers to a promoter connected to a coding sequence in such a way that the transcription of that coding sequence is controlled and regulated by that promoter. Techniques for operatively linking a promoter to a coding sequence are well known in the art; the precise orientation and location relative to a coding sequence of interest is dependent, inter alia, upon the specific nature of the promoter.

### Treatment of Steroid-Dependent Disease

In one aspect, the present invention provides a method of treating a steroid-dependent disease in a subject by providing steroid hormonal therapy to the subject while inhibiting glucocorticoid receptor activity. A problem associated with the use of steroid hormonal therapy to treat steroid-dependent disease is that steroid hormonal therapy can lead to up-regulation of glucocorticoid receptor activity, which counteracts the effect of the therapy. The present invention addresses this problem by inhibiting glucocorticoid receptor activity so that the steroid hormonal therapy can continue to be effective.

Steroid hormones are typically directly or indirectly synthesized from cholesterol, and include two different classes; corticosteroids, which are typically made in the adrenal cortex, and sex steroids, which are typically made in the gonads or placenta. Within those two classes are five types of steroid hormones, which are categorized according to the receptors to which they bind. These are glucocorticoids and mineralocorticoids, which are both corticosteroids, and androgens, estrogens, and progestogens, which are all sex steroids.

### Steroid-Dependent Disease

The present invention provides methods for treating steroid-dependent disease. Steroid-dependent disease, as used herein, refers to diseases that depend on the presence of steroid hormones. The steroid hormone upon which the disease depends varies depending on the particular disease. In various embodiments, the steroid-dependent disease can depend on glucocorticoids, mineralocorticoids, androgens, estrogens, and/or progestogens. Examples of androgen-dependent diseases steroid-dependent diseases are those that dependent on testosterone and/or 5a-dihydrotestosterone (DHT). Examples of steroid-dependent diseases include asthma, hypertension, inflammatory bowel disease (e.g., Crohn's disease and ulcerative colitis), nephritic syndrome, endometriosis, adrenal dysfunction, and some forms of cancer. As used herein, steroid-dependent disease also encompasses a disease which is normally characterized as being a steroid-dependent disease, but which has or develops steroid independence.

In some embodiments, the steroid-dependent disease is cancer. Examples of steroid-dependent cancer include bladder cancer, breast cancer, endometrial cancer, pancreatic cancer, and prostate cancer. The identification of additional types of cancer which can be steroid-dependent is ongoing. In some embodiments, the steroid upon which the cancer depends is a sex steroid. Sex steroids, also known as gonadal steroids, are steroid hormones that interact with androgen or estrogen receptors. Sex steroids include androgens such as anabolic steroids, androstenedione, dehydroepiandrosterone, dihydrotestosterone, and testosterone; estrogens such as estradiol, estriol, and estrone; and the progestogen progesterone.

As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

In some embodiments, the cancer is prostate cancer. Prostate cancer, as used herein, refers to a disease in which cancer develops in the prostate gland of the male reproductive system. Prostate cancer is classified as an adenocarcinoma, or glandular cancer, that begins when normal semen-secreting prostate gland cells mutate into cancer cells. In the initial stage of prostate cancer, small clumps of cancer cells remain confined to otherwise normal prostate glands, a condition known as carcinoma in situ or prostatic intraepithelial neoplasia (PIN), a prostate precancer. Over time these cancer cells begin to multiply and spread to the surrounding prostate tissue (the stroma), forming a tumor. While prostate cancer originates and may remain in the prostate, prostate tumor cells may develop the ability to travel in the bloodstream and lymphatic system and thus be found in other organs or tissues. Prostate cancer most commonly metastasizes to the bones, lymph nodes, rectum, and bladder. Treatment or prevention of prostate cancer, as used herein, also refers to the treatment of metastasized prostate cancer found in other organs or tissues.

Most steroid-dependent cancers become refractory after one to three years and resume growth despite therapy. Accordingly, in some embodiments, the prostate cancer is castration-resistant prostate cancer, which is also known as hormone-refractory prostate cancer or androgen-independent prostate cancer. Subjects who have castration-resistant prostate cancer are no longer responsive to castration treatment, which is a reduction of available androgen/testosterone/DHT by chemical or surgical means. However, these cancers still show reliance upon hormones for androgen receptor activation.

The presence of prostate cancer or other steroid-dependent diseases can be confirmed using a variety of techniques known to those skilled in the art. The preferred method for confirming the presence of prostate cancer is to obtain a biopsy. In a prostate cancer biopsy, a tissue samples from the prostate is typically obtained via the rectum using a biopsy gun which inserts and removes special hollow-core needles. The tissue samples are then examined under a microscope to determine whether cancer cells are present, and to evaluate the microscopic features or Gleason score of any cancer found. Additional procedures for determining whether a human subject has prostate cancer include, but are not limited to, digital rectal examination, cystoscopy, transrectal ultrasonography, ultrasound, and magnetic resonance imaging.

In case of cancer treatment, the method of steroid-dependent disease can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, and administration of immunotoxins.

### Steroid Hormonal Therapy

The present invention provides a method of improving the effectiveness of steroid hormone therapy. Steroid hormonal therapy, as used herein, refers to the use of steroid hormone deprivation to treat a steroid-dependent disease. Steroid deprivation can be caused through a variety of different methods, including administration of a steroid receptor antagonist such as an androgen receptor antagonist, administration of agents that inhibit steroidogenesis, or surgical methods such as castration or adrenal androgen ablation. The steroid deprivation is directed to decreasing the level of availability of the steroid upon which the particular steroid-dependent disease depends. Steroid deprivation can decrease the level of the steroid to a varying degree. In some embodiments, steroid deprivation decreases the level of the steroid by at least 50%, by at least 60%, by at least 70%, by at least 80%, by at least 90%, by at least 95%, or sometimes up to 100%.

In some embodiments, the steroid hormonal therapy is treatment with one or more androgen receptor antagonists. A number of androgen receptor antagonists are known to those skilled in the art. Examples of known androgen receptor antagonists include flutamide, nilutamide, bicalutamide, enzalutamide, apalutamide, galeterone, ODM-201, cyproterone acetate, megestrol acetate, chlormadinone acetate, spironolactone, canrenone, drospirenone, ketoconazole, topilutamide (fluridil), and cimetidine.

### Inhibiting Glucocorticoid Receptor Activity

In one aspect, the invention provides a method of treating steroid-dependent disease in a subject in need thereof that includes inhibiting glucocorticoid receptor activity in order to increase the effectiveness of steroid hormonal therapy. The glucocorticoid receptor (GR), also known as NR3C1 (nuclear receptor subfamily 3, group C, member 1), is the receptor to which cortisol and other glucocorticoids bind. Glucocorticoid receptor activity can be inhibited by antagonizing the glucocorticoid receptor, or by inhibiting the expression of the glucocorticoid receptor. Examples of glucocorticoid receptor antagonists include mifepristone and ketoconazole.

The inventors have also demonstrated that glucocorticoid receptor expression can be inhibited by simulating 11β-hydroxysteroid dehydrogenase-2 (11β-HSD2) activity or expression. 11β-HSD2 enzymatically converts cortisol to inactive cortisone in humans. Because 11β-HSD2 expression is suppressed in steroid-dependent disease that has become resistant to steroid hormonal therapy, treatment can be enhanced by stimulating 11β-HSD2 expression. Expression can be increased through gene therapy methods, or it can be increased by blocking 11β-HSD2 degradation. One advantage of avoiding the use of a GR antagonist is that a total and systemic GR blockade has significant adverse side effects. Sharifi, N., N Engl J Med. 370, 970-971 (2014).

While 11β-HSD2 is a preferred target for inhibiting glucocorticoid receptor expression, other enzymes involved in reinstating glucocorticoid inactivation can also be targeted in other embodiments of the invention. Examples of other enzymatic targets include 11β-HSD1, which performs a role opposite to that played by 11β-HSD2, and hexose-6-phosphate dehydrogenase (H6PD), which is responsible for making the cofactor NADPH which is required for 11β-HSD1 activity. Inhibition of H6PD or loss of its expression would reverse the directionality of 11β-HSD1, resulting in the reinstatement of the activity associated with 11β-HSD2.

The primary method for blocking 11β-HSD2 degradation is administration of a proteasome inhibitor. Proteasome inhibitors are known to those skilled in the art. Examples of proteasome inhibitors include bortezomib, carfilzomib, marizomib, ixazomib, and oprozomib. While not intending to be bound by theory, proteasome inhibitors appear to be effective for blocking 11β-HSD2 degradation as a result of inhibiting ubiquitin E3 ligase activity, which is involved in the ubiquitination of 11β-HSD2. In some embodiments, 11β-HSD2 expression is stimulated by administering an endoplasmic reticulum (ER)-associated degradation pathway inhibitor. An endoplasmic-reticulum stress pathway-associated mechanism of action has been shown to be important to the activity of proteasome inhibitors. See Ri, M., Int J Hematol. 104(3), 273-80 (2016). An example of a suitable ER-associated degradation pathway inhibitor is Eerl.

Combined treatment with steroid hormonal therapy and inhibition of glucocorticoid receptor activity can be used to provide prophylactic and/or therapeutic treatment. The active agents of the invention can, for example, be administered prophylactically to a subject in advance of the occurrence of a steroid-dependent disease. Prophylactic (i.e., preventive) administration is effective to decrease the likelihood of the subsequent occurrence of steroid-dependent disease in the subject, or decrease the severity of steroid-dependent disease that subsequently occurs. Prophylactic treatment may be provided to a subject that is at elevated risk of developing steroid-dependent disease, such as a subject with a family history of steroid-dependent disease.

Alternatively, a subject that is already afflicted by a steroid-dependent disease can be treated. A subject diagnosed as having a steroid-dependent disease is a subject in need of treatment. A subject in need of cancer treatment can be a subject who has been diagnosed as having a disorder characterized by unwanted, rapid cell proliferation. Such disorders include, but are not limited to cancers and precancerous conditions. In one embodiment of therapeutic administration, administration of the compounds is effective to eliminate the steroid-dependent disease; in another embodiment, administration of the active agents is effective to decrease the severity of the steroid-dependent disease or lengthen the lifespan of the subject so afflicted. The subject is preferably a mammal, such as a domesticated farm animal (e.g., cow, horse, pig) or pet (e.g., dog, cat). More preferably, the subject is a human.

Because the effectiveness of inhibiting glucocorticoid receptor activity is derived from its ability to overcome resistance to steroid hormonal therapy used for treatment of steroid-dependent disease, steroid hormonal therapy and inhibition of glucocorticoid receptor activity should occur close enough together in time for the inhibition of glucocorticoid receptor activity to increase the effectiveness of the steroid hormonal therapy. This can be referred to herein as being administered proximately in time. What constitutes proximately in time can vary with the metabolism of the individual, and the dose of the agents being administered. In some embodiments, proximate in time can be within 1 hour, within 6 hours, within 12 hours, or within 24 hours of administration of the other agent. In some embodiments, steroid hormonal therapy and inhibition of glucocorticoid receptor activity should occur simultaneously. However, in other embodiments, glucocorticoid receptor activity can occur proximately in time either before or after steroid hormonal therapy, or proximately in time before steroid hormonal therapy, or proximately in time after steroid hormonal therapy.

In some embodiments, inhibition of glucocorticoid receptor activity and steroid hormonal therapy are achieved using pharmaceutical agents. These active agents may be administered as a pair, or in conjunction with other drugs or nutrients, as in an adjunct therapy. The phrase “adjunct therapy” or “combination therapy” in defining use of a compound described herein and one or more other pharmaceutical agents, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

Candidate agents may be tested in animal models. The animal model should be one appropriate for the steroid-dependent disease being treated, such as prostate cancer. For example, the animal model can be one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

### Gene Therapy for Increasing 11β-HSD2 Expression

In some embodiments, 11β-HSD2 expression is stimulated using gene therapy methods. The present invention provides a method of transforming animal cells using an expression vector that includes a nucleotide sequence encoding 11β-HSD2 operatively linked to a promoter. In some embodiments, the animal cells being transformed are tumor cells. The nucleic acid sequence for HSD11B2, which encodes 11β-HSD2, is known. See Example 1, herein, in which cells are transformed to express high levels of 11β-HSD2. The nucleotide sequences used to encode 11β-HSD2 can also vary substantially as a result of the redundancy present in tRNA, in which various nucleotide triplets in the anticodon can be used to encode the same amino acid during translation. Other control elements for expression of 11β-HSD2, such as transcription factor binding sites and termination sequences, can also be included in the expression vector.

A number of transfection techniques for introducing polynucleotide sequences into animal cells are well known in the art and are disclosed herein. See, for example, Graham et al., Virology, 52: 456 (1973); Sambrook et al., Molecular Cloning, A laboratory Manual, Cold Spring Harbor Laboratories (N.Y., 1989); Davis et al., Basic Methods in Molecular Biology, Elsevier, 1986; and Chu et al., Gene, 13: 197 (1981). Such techniques can be used to introduce one or more exogenous polynucleotide sequences and their associated promoters into animal cells.

The expression vector carrying the polynucleotide sequences encoding 11β-HSD2 can be any type of expression vector suitable for the delivery of polynucleotide sequences to animal cells. A broad variety of suitable expression vectors are available for introducing polynucleotide sequences into animal cells, including plasmids, cosmids, yeast artificial chromosomes, and viral vectors. Viral vectors include retroviral-based vectors such as lentivirus, adenovirus vectors, AAV vectors, SV40 virus vectors, herpes simplex viral vectors, human cytomegalovirus vectors, Epstein-Barr virus vectors, poxvirus vectors, negative-strand RNA viral vectors such as influenza virus vectors, alpha virus vectors, and herpesvirus saimiri virus vectors, all of which have been demonstrated to be suitable for gene transfection. A particularly preferred viral vector is the adenovirus vector, which has the advantage of resulting in high levels of gene expression and being able to insert relatively large polynucleotide sequences.

In some embodiments of the invention, the expression vectors are introduced directly to a subject in vivo. Various expression vectors are particularly suitable for in vivo gene therapy applications. For example, viral vectors and non-viral vectors such as cationic liposomes, calcium phosphate precipitates, and receptor-mediated poly-lysine-DNA complexes are suitable for use in gene therapy. See, for example, Vargas et al., J Transl Med. 14(1), 288 (2016), which reviews the use of retroviral vectors and transposons for stable gene therapy.

The expression vector preferably also includes a promoter that is operatively linked to the polynucleotide sequence being expressed. The particular promoter that is employed to control the expression of a nucleic acid encoding a particular gene is not believed to be important, so long as it is capable of expressing the nucleic acid in the targeted cell. Thus, where a human cell is targeted, it is preferable to position the nucleic acid coding region adjacent to and under the control of a promoter that is capable of being expressed in a human cell.

In various instances, the human cytomegalovirus (CMV) immediate early gene promoter, the SV40 early promoter and the Rous sarcoma virus long terminal repeat can be used to obtain high-level expression of the gene of interest. The use of other viral or mammalian cellular or bacterial phage promoters which are well-known in the art to achieve expression of a gene of interest is contemplated as well, provided that the levels of expression are sufficient for a given purpose.

### Administration and Formulation

The present invention also provides pharmaceutical compositions that include agents suitable for steroid hormonal therapy and inhibition of glucocorticoid receptor activity as active ingredients, and a pharmaceutically acceptable liquid or solid carrier or carriers, in combination with the active ingredients. Any of the compounds described above as being suitable for the treatment of steroid-dependent disease can be included in pharmaceutical compositions of the invention.

The compounds can be administered as pharmaceutically acceptable salts. Pharmaceutically acceptable salt refers to the relatively non-toxic, inorganic and organic acid addition salts of the compounds. These salts can be prepared in situ during the final isolation and purification of the compound, or by separately reacting a purified compound according to formula I with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005). For example, a preferred salt form of abiraterone is abiraterone acetate.

The pharmaceutical compositions include one or more active ingredients together with one or more of a variety of physiological acceptable carriers for delivery to a subject, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The active agents can be administered as a single dose or in multiple doses. Useful dosages of the active agents can be determined by comparing their in vitro activity and their in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, inhaled, rectal, vaginal, topical, nasal, ophthalmic, or parenteral (including subcutaneous, intramuscular, intraperitoneal, and intravenous) administration.

In some embodiments, one or more of the active agents are administered orally. Formulations of the present invention suitable for oral administration may be presented as discrete units such as tablets, troches, capsules, lozenges, wafers, or cachets, each containing a predetermined amount of the active agent as a powder or granules, as liposomes containing the active compound, or as a solution or suspension in an aqueous liquor or non-aqueous liquid such as a syrup, an elixir, an emulsion, or a draught. Such compositions and preparations typically contain at least about 0.1 wt- % of the active agent.

Inhaled formulations include those designed for administration from an inhaler device. Compositions for inhalation or insufflation include solutions and suspensions in pharmaceutically acceptable, aqueous or organic solvents, or mixtures thereof, aerosols, and powders. Preferably, the compositions are administered by the oral or nasal respiratory route for local or systemic effect. Compositions in preferably pharmaceutically acceptable solvents may be nebulized by use of inert gases. Solution, suspension, or powder compositions may be administered, preferably orally or nasally, from devices that deliver the formulation in an appropriate manner. Nasal spray formulations include purified aqueous solutions of the active agent with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. In some embodiments, the active agent(s) may be incorporated into sustained-release preparations and devices.

The present invention is illustrated by the following example. It is to be understood that the particular example, materials, amounts, and procedures are to be interpreted broadly in accordance with the scope and spirit of the invention as set forth herein.

### EXAMPLES

### Example 1

### Aberrant Tumor Metabolism Enables Glucocorticoid Receptor Takeover in Enzalutamide Resistant Prostate Cancer

Metastatic prostate cancer usually responds initially to medical or surgical castration, then eventually progresses as castration-resistant prostate cancer (CRPC), which is stimulated by intratumoral synthesis of testosterone and/or 5α-dihydrotestosterone (DHT) and activation of the androgen receptor (AR). Attard et al., Lancet. 387, 70-82 (2016). Enzalutamide is a potent next-generation AR antagonist and prolongs survival for patients with metastatic CRPC. Beer T. and Tombal B., N Engl J Med. 371, 1755-6 (2014). Unfortunately, enzalutamide resistance almost always emerges, eventually leading to disease lethality.

Prostate cancer is driven by androgen stimulation of the androgen receptor (AR). The next-generation AR antagonist, enzalutamide, prolongs survival, but resistance and lethal disease eventually prevail. Emerging data suggest that the glucocorticoid receptor (GR) is upregulated in this context, stimulating expression of AR-target genes that permit continued growth despite AR blockade. However, countering this mechanism by administration of GR antagonists is problematic because GR is essential for life. The inventors show in the following example that enzalutamide treatment in models of prostate cancer and patient tissues is accompanied by a ubiquitin E3-ligase, AMFR, mediating loss of 11β-hydroxysteroid dehydrogenase-2 (11β-HSD2), which otherwise inactivates cortisol, sustaining tumor cortisol concentrations to stimulate GR and enzalutamide resistance. Remarkably, reinstatement of 11β-HSD2 expression, or AMFR loss, reverses enzalutamide resistance in mouse xenograft tumors. Together, these findings reveal a surprising metabolic mechanism of enzalutamide resistance that may be targeted with a strategy that circumvents a requirement for systemic GR ablation.

### Results

### Enzalutamide Treatment Triggers Sustained Cortisol Levels

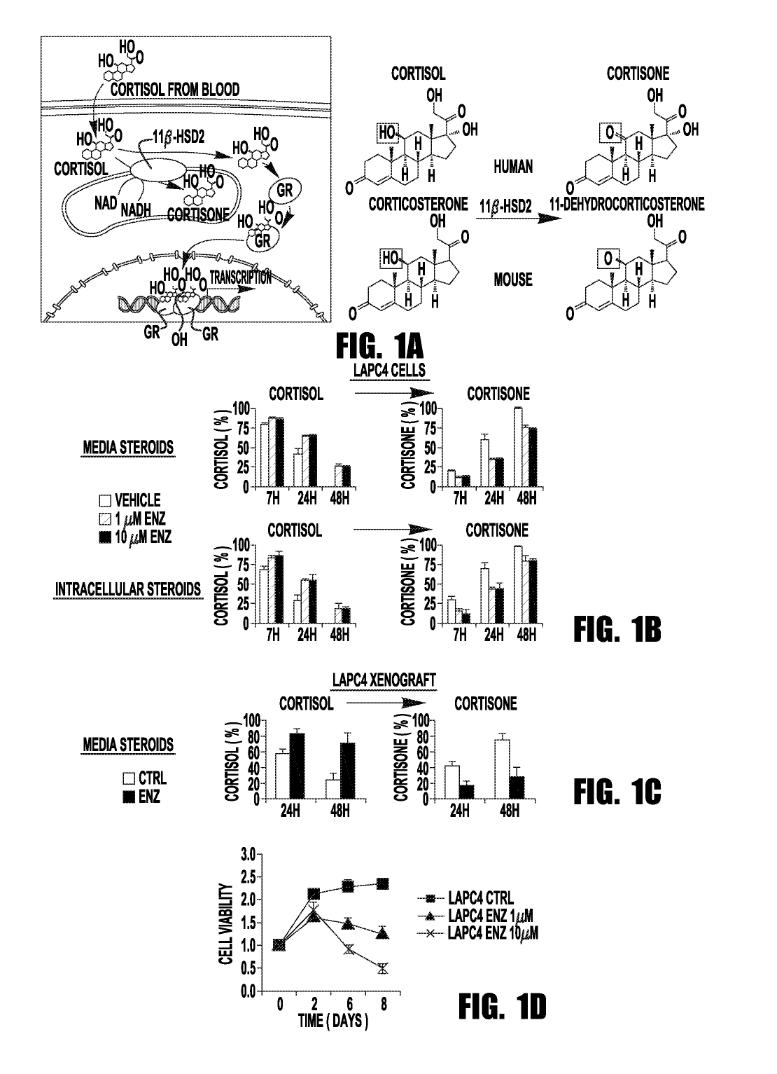

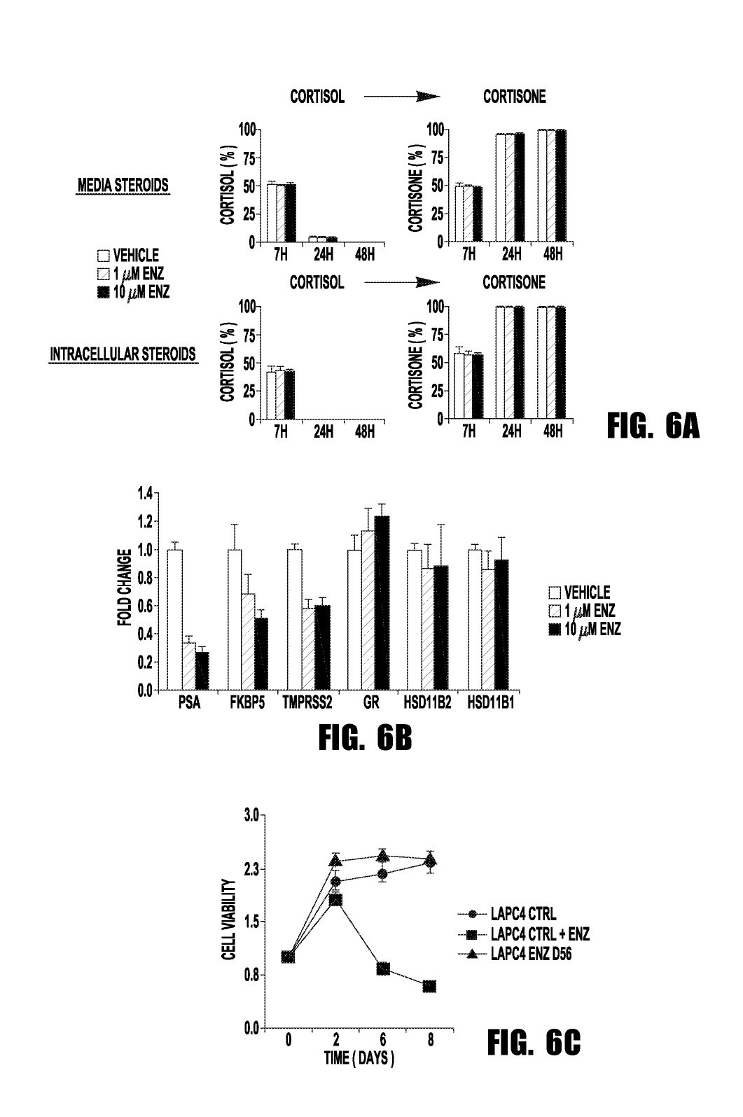

The inventors determined the effect of enzalutamide treatment on metabolic conversion of [³H]-cortisol to inactive cortisone in the LAPC4 human cell line model of CRPC. Long but not short (i.e., 24 hours) enzalutamide exposure sustains cortisol levels by retarding conversion to cortisone in cells and media (FIG. 1B and FIG. 6A). Similarly, freshly harvested CRPC xenograft tumors grown in orchiectomized mice and treated with enzalutamide (Tran et al., Science 324, 787-790 (2009)) have an impaired capability of inactivating [³H]-cortisol by conversion to cortisone, when compared to tumors from mice treated with orchiectomy alone (FIG. 1C). Enzalutamide suppresses LAPC4 viability in vitro (FIG. 1D) in association with suppression of AR-regulated genes (FIG. 6B). Growth recovers after sustained enzalutamide treatment (FIG. 6C).

### Enzalutamide Promotes 11β-HSD2 Loss

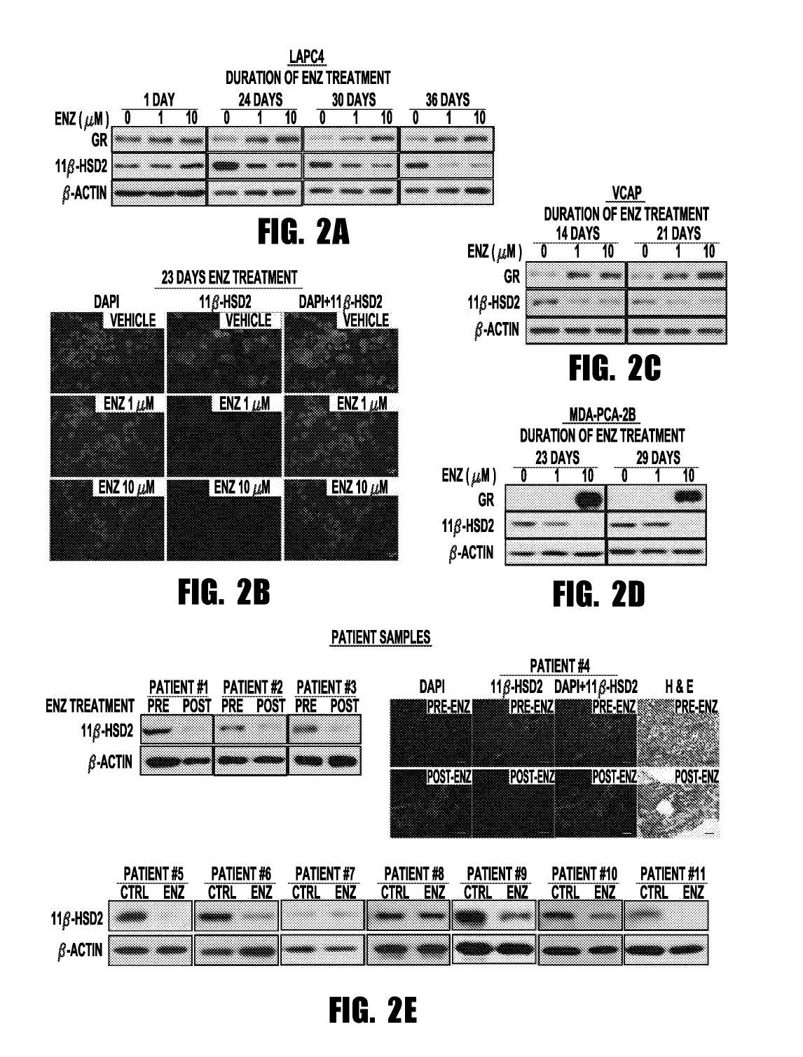

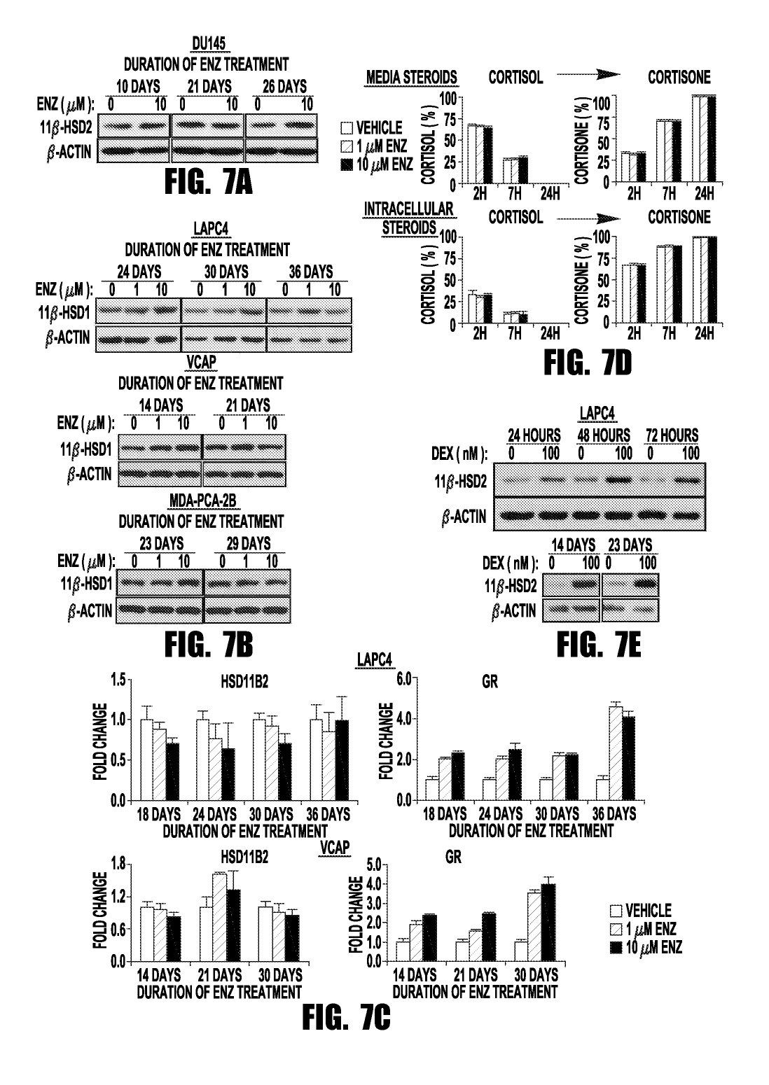

To determine the mechanism underlying the metabolic phenotype of impeded cortisol inactivation, expression of 11β-HSD2 and 11β-HSD1, which catalyzes the reverse reaction, was assessed. Loss of 11β-HSD2 protein in AR-expressing LAPC4, VCaP and MDA-PCa-2b prostate cancer cell lines (but not an AR-negative prostate cancer cell line [FIG. 7A]) was observed with enzalutamide treatment, while no consistent effect on 11β-HSD1 was detectable (FIG. 2A-D and FIG. 7B). No suppression of HSD11B2 mRNA, which encodes 11β-HSD2, was observed with enzalutamide treatment, suggesting that 11β-HSD2 protein loss is not attributable to transcriptional suppression (FIG. 7C) and no direct 11β-HSD2 antagonism by enzalutamide was observed (FIG. 7D). Importantly, 11β-HSD2 loss is not attributable to GR stimulation (FIG. 7E). To address the clinical significance of these observations, prostate tissues obtained or derived from patients and treated with enzalutamide were interrogated (FIG. 2E). Nine of 11 tissues treated with enzalutamide obtained from patients with prostate cancer (2 metastatic CRPC, 2 local prostate tissues from patients treated neoadjuvantly with enzalutamide and 7 fresh tissues treated with enzalutamide ex vivo) had loss of 11β-HSD2 with treatment. Consistent with previously published observations, GR up-regulation was observed in a subset of clinical tissues (FIG. 7F), all of which had 11β-HSD2 loss in FIG. 2E. Arora et al., Cell 155, 1309-1322 (2013). These findings demonstrate the potential clinical relevance of 11β-HSD2 protein loss for patients treated with enzalutamide.

### 11β-HSD2 Reinstatement Reverses the Metabolic Phenotype of Enzalutamide Treatment

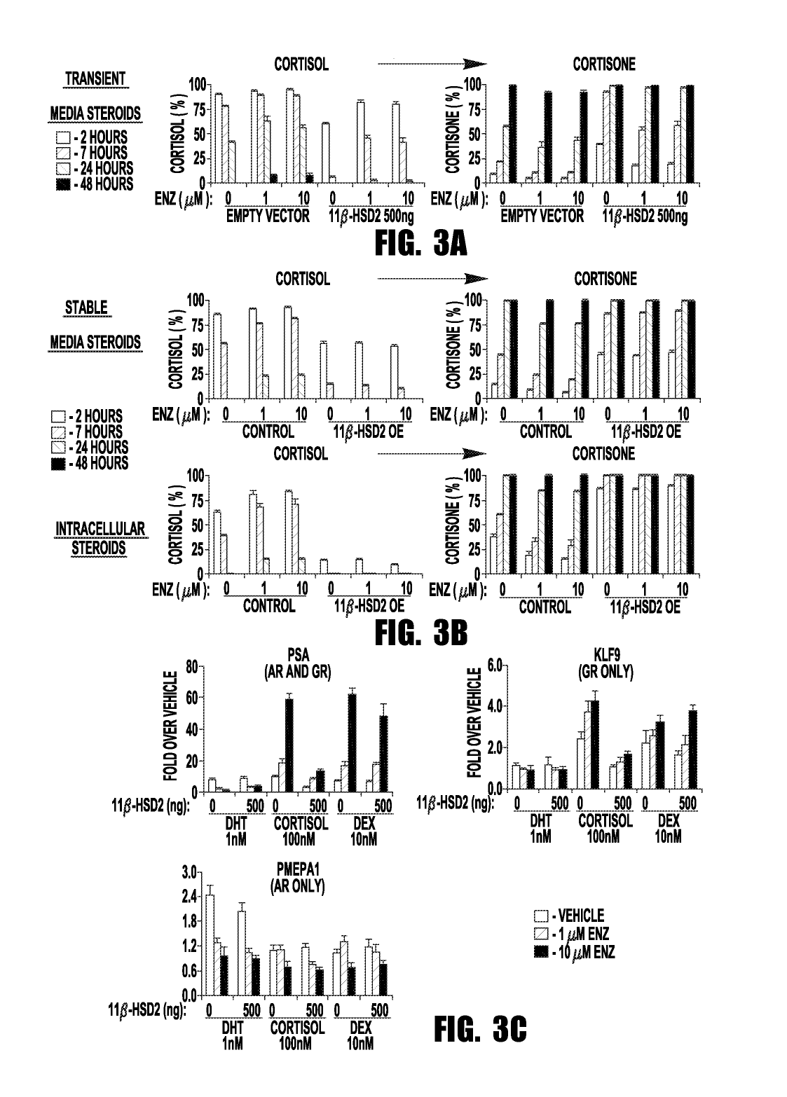

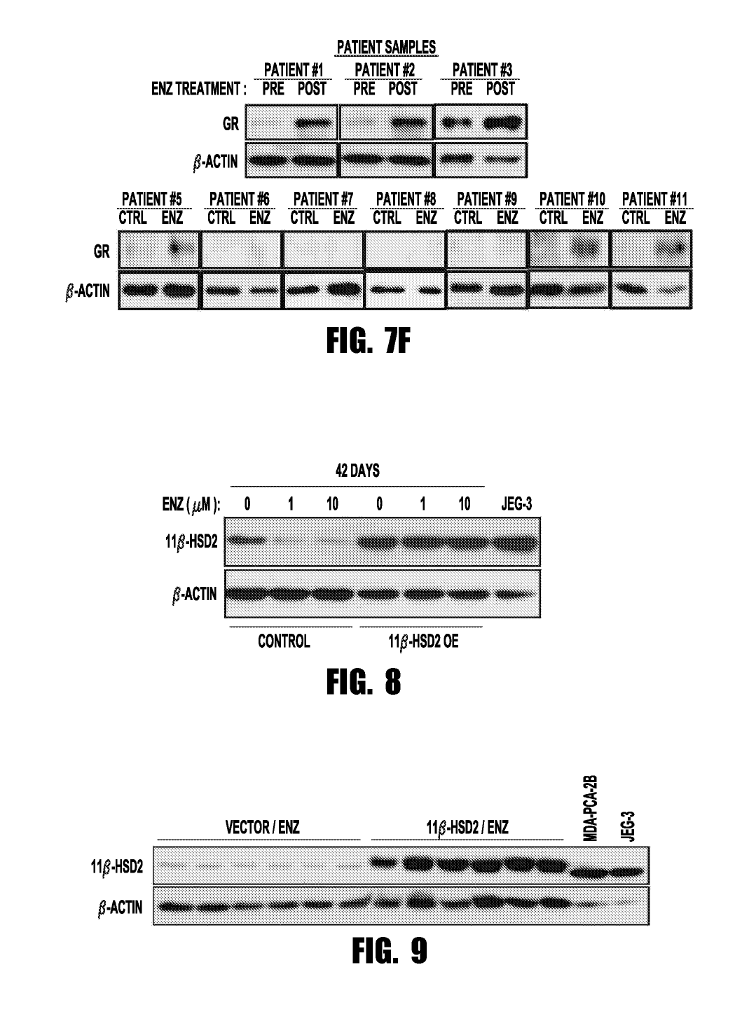

In order to investigate the metabolic effects of 11β-HSD2 replacement on cortisol levels, 11β-HSD2 was artificially expressed in the context of enzalutamide exposure (FIG. 3A-B and FIG. 8). 11β-HSD2 reinstatement with both stable expression and transient transfection studies reverted the glucocorticoid metabolic phenotype of enzalutamide treated cells back to rapid cortisol inactivation that is characteristic of enzalutamide-naive cells. Furthermore, effects of 11β-HSD2 replacement on transcription of PSA (FIG. 3C), which is regulated by AR and GR, KLF9, which is regulated by GR only and PMEPA1, which is regulated by AR only, suggest that the effects are indeed specific to glucocorticoid substrates of 11β-HSD2 (i.e., cortisol but not dexamethasone) and GR-responsive genes (i.e., PSA and KLF9).

### Reestablishing 11β-HSD2 Expression Restores Sensitivity to Enzalutamide Therapy by Depletion of Active Intratumoral Glucocorticoids

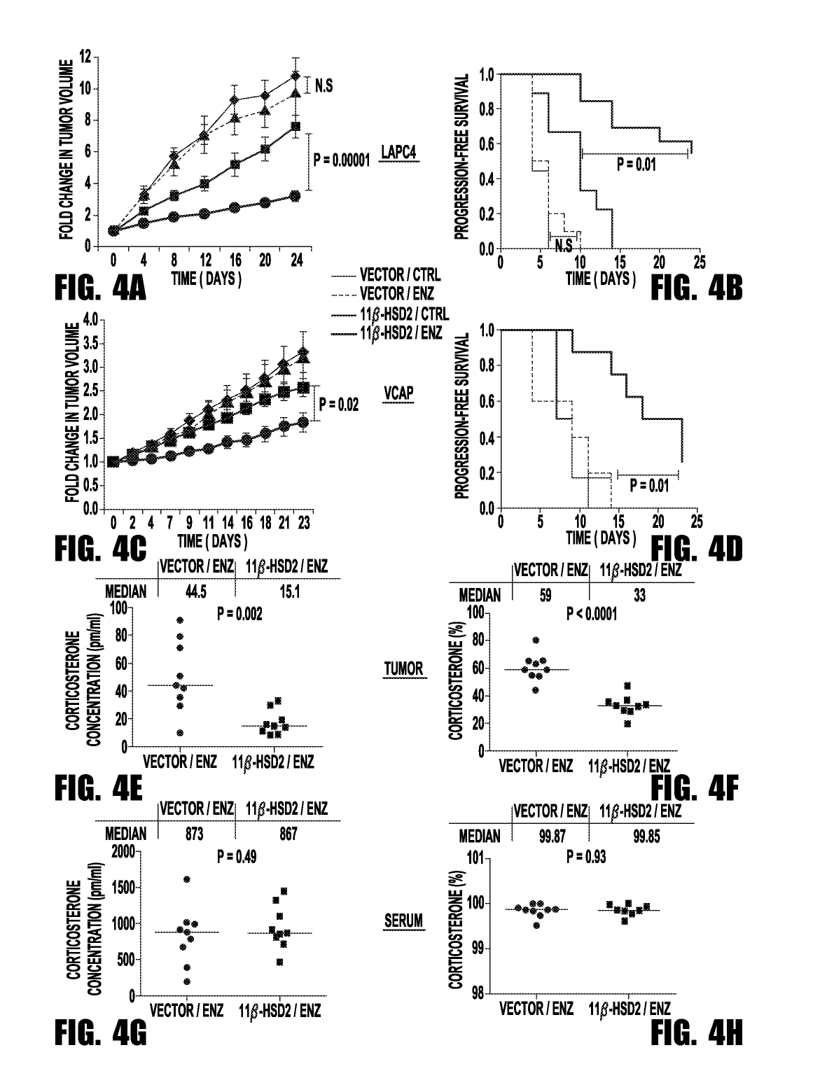

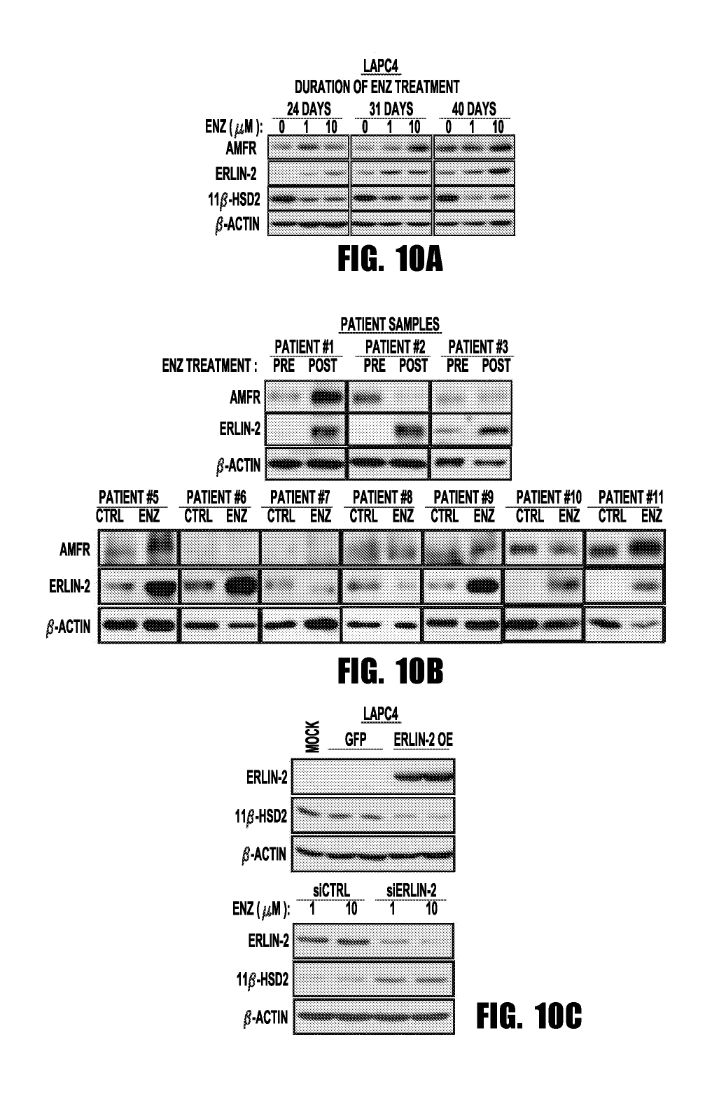

The inventors wished to determine if enzalutamide resistance is reversible with reinstatement of 11β-HSD2 expression. LAPC4 cells stably harboring a construct conferring forced 11β-HSD2 expression or vector alone (FIG. 9) were injected subcutaneously and xenograft tumors were grown in surgically orchiectomized mice that were also implanted with sustained-release DHEA pellets to mimic the human adrenal androgen milieu in CRPC. When tumors reached 100 mm³, mice in each group were randomized to enzalutamide in chow or chow alone (FIGS. 4A-B). Tumors appeared to harbor significant resistance to treatment with enzalutamide, as evidenced by growth of Vector xenografts through enzalutamide therapy. Forced 11β-HSD2 expression significantly reversed enzalutamide-resistant growth and prolonged progression-free survival. In contrast, 11β-HSD2 expression had no significant effect on untreated tumors, supporting a model in which the effect of 11β-HSD2 on tumor growth is specific to the context of resistance to potent AR antagonist therapy. Reinstatement of sensitivity to enzalutamide treatment also occurred in a second (VCaP) xenograft model of CRPC (FIGS. 4C-D). In contrast to humans, the mouse adrenal does not express 17α-hydroxylase/17,20-lyase and thus synthesizes corticosterone instead of cortisol as the dominant glucocorticoid (FIG. 1A), which is similarly inactivated to 11-dehydrocorticosterone by 11β-HSD2. To validate if reversal of enzalutamide resistance with forced 11β-HSD2 expression is accompanied by the proposed intratumoral biochemical effect of depleting biologically active tumor glucocorticoids, corticosterone concentrations were assessed in enzalutamide-treated xenograft tissues by mass spectrometry (FIG. 4E). 11β-HSD2 reinstatement depleted corticosterone concentrations by approximately two-thirds in enzalutamide-treated tumors (44.5 pmol/g in vector tumors vs. 15.1 pmol/g in 11β-HSD2 tumors; P=0.002), which otherwise harbor the capacity to metabolically sustain elevated concentrations of biologically active glucocorticoids. Tumor 11β-HSD2 expression also results in a significant decline in the percentage of tumor corticosterone (59% in vector tumors vs. 33% in 11β-HSD2 tumors; P<0.0001) when compared to the sum total of corticosterone plus 11-dehydrocorticosterone (FIG. 4F). In contrast to the effects of 11β-HSD2 expression on tumor glucocorticoids in enzalutamide treated mice, there is no significant change in serum in the absolute concentration (873 pmol/ml in vector mice vs. 867 pmol/ml in 11β-HSD2 mice; P=0.49; FIG. 4G) or percentage of corticosterone (99.87% in vector mice vs. 99.85% in 11β-HSD2 mice; P<0.93; FIG. 4H).

### AMFR is Required for 11β-HSD2 Ubiquitination, the Metabolic Phenotype of Retarded Glucocorticoid Inactivation and Enzalutamide Resistance

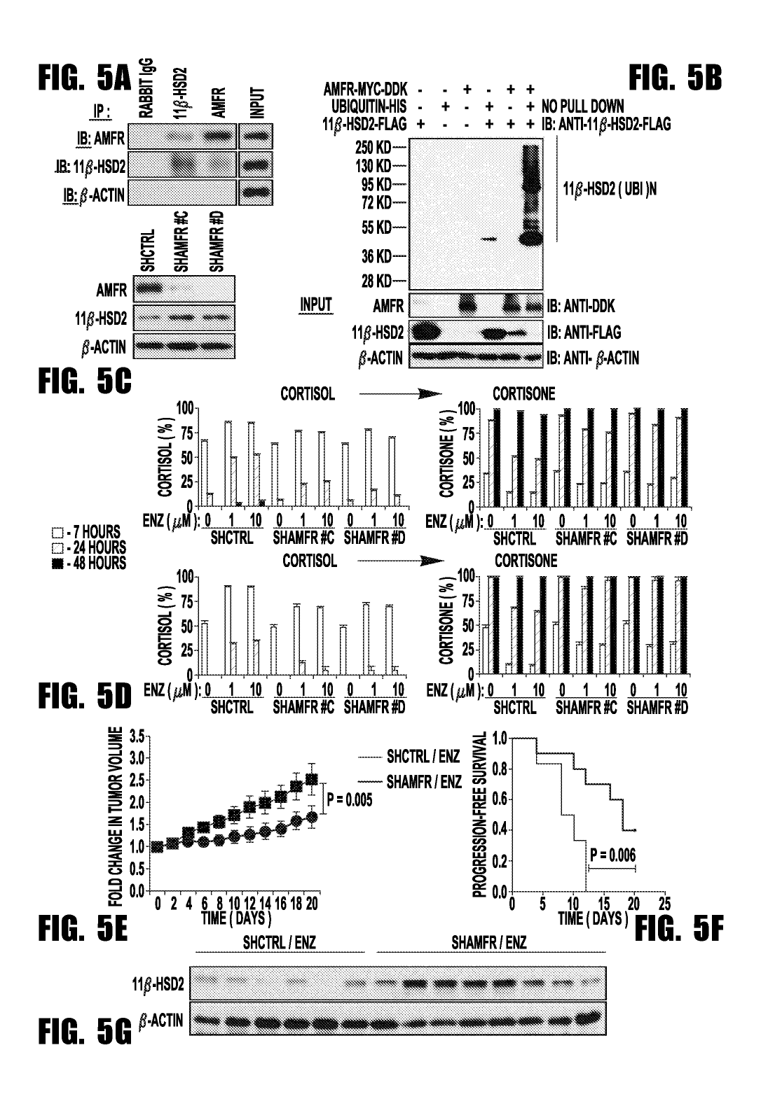

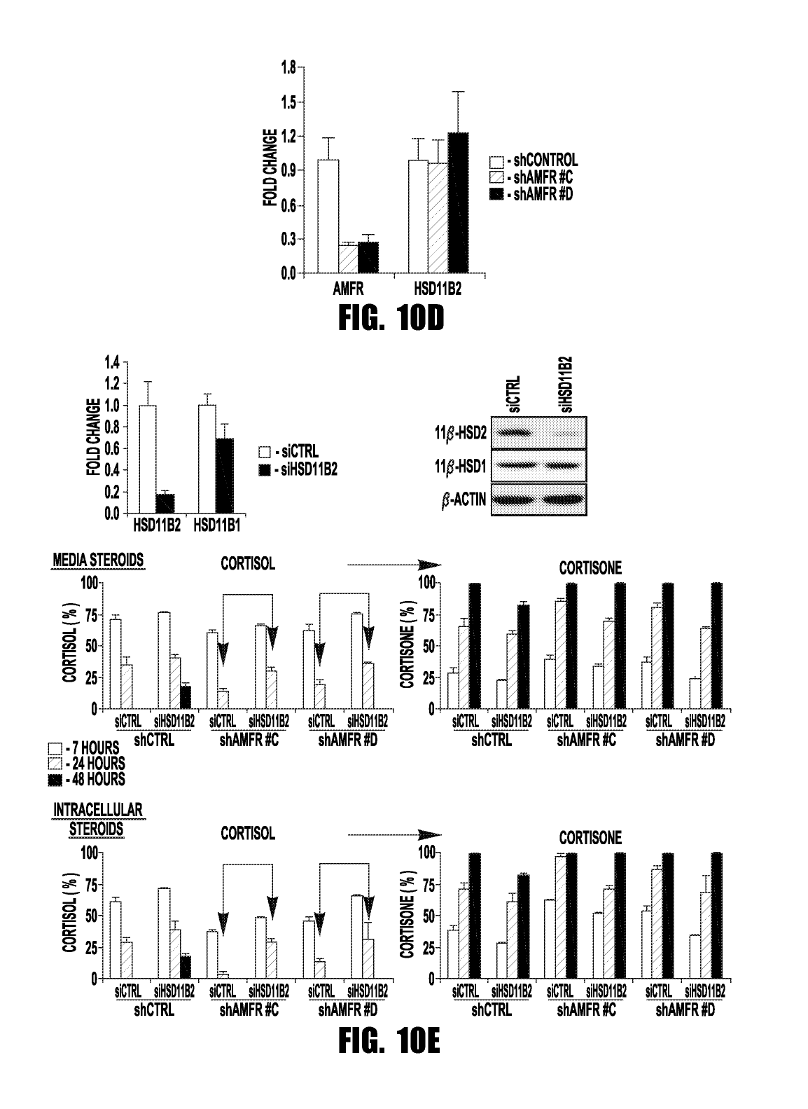

The inventors previously described the role of AMFR, a ubiquitin E3-ligase in the endoplasmic reticulum associated degradation pathway, in regulation of another steroidogenic enzyme 3β-hydroxysteroid dehydrogenase-1. Chang et al., Cell 154, 1074-1084 (2013). As 11β-HSD2 is also located in the endoplasmic reticulum, they hypothesized that AMFR is required for enzalutamide-induced loss of 11β-HSD2 protein. A physical association between 11β-HSD2 and AMFR is supported by immunoprecipitation of AMFR, followed by immunoblot for 11β-HSD2, as well as immunoprecipitation of 11β-HSD2, followed by immunoblot for AMFR (FIG. 5A). Expression of 11β-HSD2, Ubiquitin-His, and AMFR-Myc-DDK, followed by Ni-agarose pull-down and anti-11β-HSD2 immunoblot demonstrates the AMFR-dependence of 11β-HSD2 ubiquitination (FIG. 5B). Enzalutamide treatment did not consistently increase AMFR expression (FIG. 10A-B). However, Erlin-2, which enables the AMFR-associated endoplasmic reticulum-associated degradation pathway (ERAD), was more consistently up-regulated, including in 8 of 11 patient tissues (FIG. 10A-C). The functional consequence of 11β-HSD2/AMFR interaction and 11β-HSD2 ubiquitination is evidenced by genetically silencing AMFR with stable shRNA expression, which promotes an increase in 11β-HSD2 protein (FIG. 5C) but not transcript (FIG. 10D). Furthermore, the enzalutamide-induced metabolic phenotype that sustains cortisol concentrations by way of retarded inactivation is reversed with genetic ablation of AMFR (FIG. 5D). Silencing both 11β-HSD2 and AMFR with enzalutamide treatment negates the effect of genetically silencing AMFR alone, suggesting that the effect of AMFR is mediated through 11β-HSD2 (FIG. 10E). Finally, the functional relevance and requirement for AMFR expression on enzalutamide resistance is suggested by suppressed xenograft growth and prolonged progression-free survival in enzalutamide-treated xenografts with stable AMFR knockdown (FIGS. 5E-F). Tumors with AMFR knockdown were confirmed to have sustained 11β-HSD2 protein expression, thus impairing enzalutamide resistance (FIG. 5G). Together, these findings suggest a model in which a physical association between AMFR and 11β-HSD2 enables enzalutamide to promote loss of 11β-HSD2, resulting in sustained cortisol concentrations that promote GR stimulation.

### Discussion

The findings reveal a metabolic mechanism that is co-opted along with GR upregulation to stimulate enzalutamide resistance in prostate cancer. These findings indicate that systemic availability of GR agonists represents only one side of the coin for tumor GR stimulation in the setting of enzalutamide resistance. Local metabolic regulation of ligand availability by the tumor serves as the other face of the coin and can either oppose the effects of systemic glucocorticoids by spurring enzymatic inactivation, or instead allow unimpeded access to the tumor tissue, enabling sustained GR stimulation to promote tumor progression.

Glucocorticoid administration has long been recognized to have a therapeutic effect in CRPC. This effect is likely in large part indirect and attributable to suppression of adrenal androgen production. Attard et al. J Clin Endocrinol Metab., 97, 507-16 (2012). However, it has been recently recognized that GR stimulation may also contribute to prostate cancer progression. The inventors' findings suggest yet another consideration that adds to the complexity of glucocorticoid signaling in prostate cancer, namely susceptibility of the administered glucocorticoid to metabolic inactivation, that is likely relevant to the increased GR expression that may occur alongside enzalutamide resistance and allow direct tumor-promoting effects of glucocorticoids in CRPC. For example, prednisolone is inactivated by 11β-HSD2 to prednisone but dexamethasone is generally thought to be impervious to inactivation by 11β-HSD2. This may be even more important prior to enzalutamide therapy and consequent suppression of 11β-HSD2 loss, because some data suggest that baseline GR expression prior to enzalutamide treatment, where 11β-HSD2 expression is intact, may be associated with enzalutamide resistance.

Metabolic regulation of GR stimulation by the tumor might also be a tumor-specific therapeutic vulnerability. The data raise the possibility that blocking 11β-HSD2 protein loss, for example by blocking AMFR, or reinstatement of 11β-HSD2 expression in the tumor may be an appropriate strategy to reverse enzalutamide resistance without affecting systemic availability of glucocorticoids and resultant associated toxicities. Blocking AMFR may also increase 3βHSD1 protein, sustaining androgen synthesis. However, the in vivo studies with AMFR knockdown suggest that in the context of enzalutamide treatment, the net effect of AMFR ablation is therapeutic, probably because the AR ligand binding domain remains mainly occupied by enzalutamide and glucocorticoid signaling is a major driver of tumor progression.

Finally, the findings may have general relevance to steroid-dependent disease processes that use alternative steroid receptors. For example, in addition to the involvement of GR in prostate cancer, GR and AR have been implicated in subtype-specific breast cancer progression. Ni et al., Cancer Cell 20, 119-131 (2011). The results suggest that a switch in steroid receptors that drives disease processes more broadly may be accompanied by perturbed local metabolic regulation of the availability of ligands that stimulate steroid receptor activation.

### Materials and Methods

### Cell Lines

LAPC4 was a generous gift from Dr. Charles Sawyers (Memorial Sloan Kettering Cancer, New York, N.Y.), which was maintained in Iscove's modified Dulbecco's medium (IMDM) with 10% fetal bovine serum and incubated in a 5% CO₂ humidified incubator. VCaP was purchased from American Type Culture Collection (ATCC), which was cultured in Dulbecco's Modified Eagle Medium (DMEM) containing 10% fetal bovine serum and incubated in an 8% CO₂ humidified incubator. MDA-PCa-2b was purchased from ATCC, which was cultured in BRFF-HPC1TM (Athena ES) containing 20% fetal bovine serum and incubated in a 5% CO₂ humidified incubator. FCIV1-11β-HSD2-FLAG was used to generate the LAPC4 and VCaP stable cell line expressing human 11β-HSD2 by using a lentiviral system. The viral packaging and infection was performed as previous described. Chang et al., Proc Natl Acad Sci U.S.A., 108, 13728-33 (2011) Briefly, 293T cells (ATCC) were cotransfected with 10 μg each of FCIV1-11β-HSD2-FLAG, pMD2.G, and psPAX2 vector for 48 hours to package the virus. Next, LAPC4 and VCaP cells were infected with the virus for 24 hours with addition of polybrene (6 mg/ml), followed by selection with 2 g/ml puromycin for ˜2 weeks. The AMFR knockdown LAPC4 stable cell line was established by employing the pGFP-C-shLenti vectors contain AMFR shRNA sequences (OriGene), The viral packaging, infection as well as selection procedures were carried out as described herein. Enzalutamide (Enz) was obtained courtesy of Medivation (San Francisco, Calif.). All Enz and vehicle treated cells were maintained in medium containing 10 nM DHEA. Cell lines were authenticated by DDC Medical and routinely screened for mycoplasma contamination as described. Li et al., Nature 523, 347-351 (2015).

### Cortisol Metabolism

Cell line metabolism. Cells (˜10⁶ cells per well) were plated and maintained in 12 well plates coated with poly-DL-ornithine (Sigma-Aldrich) for ˜24 hours and then treated with [³H]-cortisol (1,000,000 counts per minute (c.p.m.) per well; PerkinElmer) and non-radiolabeled cortisol (100 nM final concentration). After incubation for the indicated time points, both media and cells were collected. Briefly, 300 μl media was collected; the cells were scraped and centrifuged at 10,000×g for 2 minutes twice to remove all the media, then the cell pellets were resuspended with 300 μl PBS. Collected media and cells were incubated with 300 units of β-glucuronidase (Helix pomatia; Sigma-Aldrich) at 65° C. for at least 2 hours, extracted with 600 μl 1:1 ethyl acetate:isooctane, and concentrated under a nitrogen stream.

Xenograft metabolism. LAPC4 cells (˜10⁷) were injected subcutaneously with Matrigel (Corning) into surgically orchiectomized NSG mice that were implanted with DHEA pellets (5 mg/pellet, 90-day sustained-release, Innovative Research of America). Fresh xenografts were harvested and ˜40 mg xenograft tissues were minced, and cultured in IMEM with 10% FBS at 37° C. with a mixture of [³H]-cortisol and non-radiolabeled cortisol. Aliquots of media were collected at the indicated time points, steroids were extracted and concentrated as described above.

For HPLC analysis, the concentrated samples were dissolved in 50% methanol and injected on a Breeze 1525 system equipped with model 717 plus autoinjector (Waters Corp.). Steroid metabolites were separated by a Luna 150×4.6 mm, 3 μM C18 reverse-phase column (Phenomenex) using methanol/water gradients at 30° C. The column effluent was analyzed using a β-RAM model 3 in-line radioactivity detector (IN/US Systems, Inc.) using Liquiscint scintillation mixture (National Diagnostics). All metabolism studies were performed in duplicate and repeated in independent experiments.

### Gene Expression and Immunoblot

LAPC4 cells were treated with Enz for 36 days and then seeded into 12 well plates coated with poly-DL-ornithine at 50% confluence. After incubation overnight, the cells were transfected with 11β-HSD2-FLAG plasmid for 48 hours by using the TranslT®-2020 Transfection Reagent (Mirus) according to the protocol provided by the manufacturer, then maintained in phenol-red-free medium with 5% Charcoal:Dextran-stripped FBS for 48 hours before being treated with the indicated drugs. Total RNA was extracted with a GenElute Mammalian Total RNA miniprep kit (Sigma-Aldrich) and 1 μg RNA was reverse-transcribed to cDNA with the iScript cDNA Synthesis Kit (Bio-Rad). An ABI 7500 Real-Time PCR machine (Applied Biosystems) was used to perform the qPCR analysis, using iTaq Fast SYBR Green Supermix with ROX (Bio-Rad) in 96-well plates at a final reaction volume of 10 μl. The qPCR analysis was carried out in triplicate with suitable primers. Each mRNA transcript was quantitated by normalizing the sample values to RPLP0 and to vehicle treated cells (for steroid treated cells). All gene expression studies were repeated in independent experiments.

For protein analysis, immunoblots were performed as described previously. Li et al., J Clin Endocrinol Metab 98, E1189-1197 (2013). Briefly, total cellular protein was extracted with ice cold RIPA lysis buffer (Sigma-Aldrich) containing protease inhibitors (Roche). 30-50 μg protein was separated by 8% SDS-PAGE gel and then transferred to a nitrocellulose membrane (Millipore). After incubating with the anti-11β-HSD2 antibody (Santa Cruz; 1:3000), anti-11β-HSD1 antibody (Santa Cruz; 1:1000), GR antibody (BD Biosciences; 1:1000), anti-Erlin-2 antibody (Cell Signaling Technology: 1:1000) or anti-AMFR antibody (Proteintech; 1:1000) overnight at 4° C., the appropriate secondary antibody was incubated for 1 hour at room temperature. The chemiluminescent detection system (Thermo Scientific) was used to detect the bands with peroxidase activity. An anti-β-actin antibody (Sigma-Aldrich; 1:5000) was used as a control for sample loading.

### Gene Expression and Knockdown

Gene expression. The day before transfection, LAPC4 cells were plated into 12 well plates coated with poly-DL-ornithine (˜7×10⁵ cells/well). On the day of transfections, an Erlin-2 expressing plasmid, Erlin-2-Myc-DDK-tagged (OriGene) or 11β-HSD2-expressing plasmid FCIV1-11β-HSD2-FLAG was introduced into the cells with Lipofectamine® 3000 Reagent (Life Technology). After 48 hours, the cells were collected and used for the detection of 11β-HSD2 and Erlin-2 by immunoblot, or treated with Enz for 24 hours to determine the effects on cortisol metabolism by HPLC as described herein.

Gene knockdown. LAPC4 cells were seeded into 12 well plates coated with poly-DL-ornithine at 60-80% confluence. After incubation overnight, the cells were transfected with siRNA following the Lipofectamine® RNAiMAX Reagent (Life Technology) protocol provided by the manufacturer for 48 hours. Extracted RNA and cell lysates were used for analyses by qPCR and immunoblot. For 11β-HSD2 knockdown, the experiments were performed with Dhamarcon SMARTpool: ON-TARGETplus HSD11B2 siRNA, L-008983-00-0005 or ON-TARGET plus Non-targeting Pool, #D-001810-10-05 with a final concentration of 25 nM siRNA. For Erlin-2 knockdown, the siRNA sequence, see Huber et al., J. Cell Biol. 203, 427-436 (2013).

Cell Viability Assay

LAPC4 cells or the long-term Enz treated LAPC4 cells were plated in triplicate in 96 well plates coated with poly-DL-ornithine and incubated overnight, then treated with Enz and assayed in triplicate at the time points indicated using CellTiter-Glo (Promega). Viability is normalized to day 0.

### Co-Immunoprecipitation

The interaction between 11β-HSD2 and AMFR was analyzed using the Pierce Classic Magnetic IP/Co-IP Kit (Thermo Scientific) following the protocol provided by the manufacturer. Briefly, ˜10⁷ LAPC4 cells were lysed in 1 ml Pierce IP Lysis/Wash Buffer with protease inhibitors added fresh, on ice for 1 hour. The cell lysates were centrifuged at 12,000×g for 15 minutes at 4° C. 1-2 mg of protein was pre-cleaned with 30 μl pre-cleaned Protein A/G PLUS-Agarose (Santa Cruz) and 1 μg rabbit IgG (Millipore) for 1 hour and then incubated with rabbit IgG (3 μg), 11β-HSD2 antibody (4 μg) or AMFR antibody (3 μg) overnight at 4° C. The antibody/antigen/bead complex was washed with ice-cold Pierce IP Lysis/Wash Buffer containing protease inhibitors adequately and denatured in 40 μl freshly prepared 1× Lane Marker Sample Buffer at room temperature for 30 minutes with mixing. 20 μl IP products were used for the subsequent protein separation and detection of 11β-HSD2 or AMFR using their antibodies by immunoblot.

### Mouse Xenograft Studies

All mouse studies were performed under a protocol approved by the Institutional Animal Care and Use Committee (IACUC) of the Cleveland Clinic Lerner Research Institute. All NSG male mice (6-8 weeks old) were purchased from the Jackson Laboratory and the number of mice used in this study was based on previously published mouse xenograft studies by our lab that determined effects of steroid pathway inhibition/augmentation on xenograft growth. Mice were surgically orchiectomized and implanted with DHEA pellets to mimic human adrenal DHEA production in patients with CRPC. 1 week later, mice were prepared for cell injections.

For the evaluation of the 11β-HSD2 role in reversing enzalutamide resistance, either 10⁷ vector control or 10⁷ 11β-HSD2 overexpressing LAPC4 or VCaP cells (100 μl in 50% matrigel and 50% growth media) were subcutaneously injected into mice. When tumors reached 100 or 150 mm³ (length×width×height×0.52), for LAPC4 and VCaP xenografts, respectively, the mice were arbitrarily divided into 2 groups each for vector and 11β-HSD2 overexpressing cells: Enz diet 62.5 mg/kg and or chow alone groups. Tran et al., Science 324, 787-790 (2009). Based on the daily chow consumption, approximately 0.3125 mg Enz was consumed per mouse per day. Enz in chow and chow alone were obtained from Medivation. Tumor volume was measured every other day, and progression-free survival was assessed as time to 3-fold (LAPC4) or 1.5-fold (VCaP) increase in tumor volume (from 100 or 150 mm³) from the time Enz or chow alone was initiated. The number of mice in the LAPC4 Vector/Ctrl, Vector/Enz, 11β-HSD2/Ctrl and 11β-HSD2/Enz groups were 9, 9, 10, and 11, respectively. The number of mice in the VCaP Vector/Ctrl, Vector/Enz, 11β-HSD2/Ctrl and 11β-HSD2/Enz groups were 6, 6, 5, and 8, respectively. Numbers of mice in each treatment group were determined by those that survived surgical procedures and had reached a tumor volume to initiate treatment.

For evaluation of the role of AMFR in reversing enzalutamide resistance, either 10⁷ control or 10⁷ AMFR knockdown LAPC4 cells (100 μl in 50% matrigel and 50% growth media) were subcutaneously injected into mice. The remaining procedures were performed as described above. The number of mice in the LAPC4 shCtrl/Enz and shAMFR/Enz groups were 6, and 10, respectively. Protein levels of 11β-HSD2 in the shCtrl and control or shAMFR LAPC4 xenografts were analyzed by immunoblot. Briefly, ˜40-50 mg xenograft tissue was minced into pieces and then added into soft tissue homogenizing CK14 tubes (Betin Technologies) with 150 μl RIPA buffer containing protease inhibitors. Xenograft tissues were homogenized with a homogenizer (Minilys, Betin Technologies) 6 times (40 seconds each time) at the highest speed. Tubes were incubated on the ice for 5-10 minutes between each homogenization to cool lysates. The lysates were then centrifuged for 30 minutes at 15,000×g and the supernatants were used for immunoblot analysis.

Immunofluorescence Staining

LAPC4 cells treated with Enz or vehicle for 23 days were seeded into chamber slides (BD Biosciences) coated with poly-DL-ornithine at 60% confluence. After overnight culture, cells were washed with PBS and fixed with ice cold methanol for 15 minutes and the methanol was washed with PBS. Before applying primary antibodies, nonspecific binding sites were blocked with blocking buffer (Protein Block Serum Free, Dako). Anti-11β-HSD2 antibody (Santa Cruz), diluted at 1:300 with Antibody Diluent (Dako), was applied for incubation overnight at 4° C. After being rinsed with PBS, the slides were probed with Alexa Fluor® 594 conjugated secondary antibody (goat anti-rabbit, Thermo Scientific) for 45 minutes at room temperature. VECTASHIELD HardSet™ Mounting Medium (Vector Laboratories) was used to mount the slides and counterstain the nucleus with DAPI.

### Human Tissue Studies

All deidentified human tissues were obtained using institutional review board (IRB)-approved protocols. Pre- and post-Enz lymph node tissues were obtained from CT-guided biopsy of metastatic CRPC from Cleveland Clinic (Patient #3) and Dana-Farber Cancer Institute (Patient #4). Pathologic identification of tumor was done by an expert prostate cancer pathologist. Staining of tissues from Patient #4 was done with frozen section slides that were air dried at room temperature for 5 minutes, followed by rehydration with PBS. Immunofluorescence staining was performed as described above. H&E staining was completed by the imagine core of Biomedical Engineering Department in Lerner Research Institute of Cleveland Clinic.

Paired pre-Enz treatment and post-Enz treatment tissues from Patient #1 and Patient #2 were obtained from patients with localized prostate cancer treated with Enz plus ADT for 2 months prior to the second biopsy in a clinical trial (NCT02064582) at the University of Texas Southwestern Medical Center. Biopsies were obtained using image-guidance with a Koelis Urostation. The biopsy cores were minced into pieces and then added into Soft tissue homogenizing CK14 tubes (Betin Technologies) with 100 μl 6M Urea buffer containing protease inhibitors (Sigma Aldrich). Tissue homogenization and immunoblot analysis were performed as described previously.

Seven fresh prostate tissue cores (60-80 mg) from Patients #5-#11 were obtained from the peripheral zone of radical prostatectomy specimens at Cleveland Clinic, confirmed to have tumor in or in close proximity to cores by an expert prostate cancer pathologist, minced and aliquoted to two parts. One was treated with 10 nM DHEA plus vehicle, and the other was treated with 10 nM DHEA plus 10 μM Enz. Both tissues were maintained in 3 ml DMEM containing 10% fetal bovine serum and incubated in a 5% CO₂ humidified incubator. After 4 days of culture, 2 more ml medium with DHEA plus either vehicle or Enz was added into each part. The tissues were collected after 7-8 days treatment. The same procedures were performed as described above for protein extraction and immunoblot.

### Mass Spectrometry

Xenograft analysis. At least 24 mg tumor tissue (n=18) was homogenized with 1 ml LC-MS grade water (Fisher) by using homogenizer. The mixture was then centrifuged. 800 μl of the supernatant was transferred to a glass tube, followed by the addition of 80 μl of 10 ng/ml internal standard (corticosterone-d8) (Steraloids). The steroids and the internal standard were extracted with methyl tert butyl ether (Across) evaporated to dryness under N₂ then reconstituted with 500 μl of 50% methanol.

Mouse serum analysis. At the endpoint of the xenograft study, mouse serum was collected. 20 μl of mouse serum and internal standard (corticosterone-d8) were precipitated with 200 μl methanol. After centrifugation, the supernatant was transferred to HPLC vials prior to mass spectrometry analysis.

The LC-MS/MS system contains an ultra-pressure liquid chromatography system (Shimadzu Corporation, Japan) which is consisted of two LC-30AD pumps, a DGU-20A5R degasser, a CTO-30A column oven, SIL-30AC autosampler, and a system controller CBM-20A and coupled with a Qtrap 5500 mass spectrometer (AB Sciex). Data acquisition and processing were performed using Analyst® software (version 1.6.2) from ABSciex.

Steroids were ionized using electrospray ionization in positive mode. Quantification of analytes was performed using multiple reaction monitoring. The mass transitions for corticosterone, 11-dehydrocorticosterone, and internal standard are 347.3/121.0, 345.3/121.0, and 355.3/125.0, respectively. Separation of steroids was achieved using a Zorbax Eclipse plus C18 column (Agilent) using mobile phase consisting of (A) 0.2% formic acid in water and (B) 0.2% formic acid in (methanol:acetonitrile, 60:40) with a gradient program at a flow rate of 0.2 ml/min. Sample injection volume was 10 μl.

### Ubiquitination Assay

Experiments were conducted as previously described, with minor modifications. Chang et al., Cell 154, 1074-1084 (2013). Briefly, HEK293T were transfected with the following plasmids: FCIV1-11β-HSD2-FLAG, pcDNA3-6xHis-ubiquitin and pLenti-AMFR-Myc-DDK (OriGene) for 36 hours. Transfected cells were collected by scraping with ice-cold PBS. Cells pellets were suspended in 200 μl PBS. For input analysis, 20 μl of cell suspension was pelleted and lysed with RIPA lysis buffer, followed by immunoblot analyses with anti-DDK (OriGene, 1:1000), anti-FLAG (Sigma-Aldrich, 1:1000) and anti-β-actin antibodies. The remaining cells were lysed with 4 ml lysis buffer (6 M guanidine-HCl, 0.1M Na₂HPO₄/NaH₂PO₄, 0.01MTris/HCl, pH 8.0, 10 mM imidazole, and 10 mM β-mercaptoethanol) and sonicated to reduce the viscosity. Protein complexes were pulled down by incubation with 30 μl Ni NTA magnetic agarose beads (QIAGEN) at room temperature for 2 hours and then successively washed with the buffer series: (1) 6 M guanidine-HCl, 0.1M Na₂HPO₄/NaH₂PO₄, 0.01M Tris/HCl, pH 8.0, 10 mM imidazole, and 10 mM β-mercaptoethanol; (2) 8 M Urea, 0.1 M Na₂HPO₄/NaH₂PO₄, 0.01 M Tris/HCl, pH 8.0, 20 mM imidazole, 10 mM β-mercaptoethanol plus 0.2% Triton X-100; (3) 8 M urea, 0.1 M Na₂HPO₄/NaH₂PO₄, 0.01 M Tris/HCl, pH 6.3, 10 mM β-mercaptoethanol (buffer A), 40 mM imidazole plus 0.4% Triton X-100, twice; (4) buffer A with 20 mM imidazole plus 0.2% Triton X-100; (5) buffer A with 10 mM imidazole plus 0.1% Triton X-100. After the washes, the protein complexes were eluted with 30 μl 2X SDS sample buffer containing 400 mM imidazole and 20 μl elution was then used for immunoblot analysis with anti-11β-HSD2 antibody (Santa Cruz; 1:3000).

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing possible mechanisms through with the compounds are effective, the inventors are not bound by theories described herein. The invention is not limited to the exact details shown and described, for variations obvious to one skilled in the art will be included within the invention defined by the claims.

1. A method of treating a steroid-dependent disease in a subject by providing steroid hormonal therapy to the subject while inhibiting glucocorticoid receptor activity.

2. The method of claim 1, wherein glucocorticoid receptor activity is inhibited by stimulating 11β-HSD2 expression.

3. The method of claim 1, wherein the steroid-dependent disease is steroid-dependent cancer.

4. The method of claim 3, wherein the method further comprises the step of ablating the cancer.

5. The method of claim 3, wherein the steroid-dependent cancer is prostate cancer.

6. The method of claim 5, wherein the prostate cancer is metastatic prostate cancer.

7. The method of claim 5, wherein the prostate cancer is castration-resistant prostate cancer.

8. The method of claim 1, wherein the steroid hormonal therapy is treatment with an androgen receptor antagonist.

9. The method of claim 8, wherein the androgen receptor antagonist is enzalutamide.

10. The method of claim 2, wherein 11β-HSD2 expression is stimulated by blocking 11β-HSD2 degradation using a proteasome inhibitor.

11. The method of claim 10, wherein the proteasome inhibitor is selected from the group consisting of bortezomib, carfilzomib, marizomib, ixazomib, and oprozomib.

12. The method of claim 2, wherein 11β-HSD2 expression is stimulated by administering an endoplasmic reticulum (ER)-associated degradation pathway inhibitor.

13. The method of claim 2, wherein 11β-HSD2 expression in the subject is increased by gene therapy.
